(19) United States
(12) Patent Application Publication
(10) Pub. No.: US 2022/0143259 A1
(43) Pub. Date: May 12, 2022

(54) AIR STERILIZATION APPARATUS AND METHOD FOR VEHICLE

(71) Applicant: AVRA Medical Robotics, Inc., Orlando, FL (US)

(72) Inventors: Barry Cohen, Fort Lauderdale, FL (US); Alexandre Clug, Jupiter, FL (US); Ettore Tomasetti, Delray Beach, FL (US)

(21) Appl. No.: 17/410,714

(22) Filed: Aug. 24, 2021

Related U.S. Application Data
(60) Provisional application No. 63/069,642, filed on Aug. 24, 2020.

(51) Int. Cl. A61L 9/20; B60H 3/00

(52) U.S. Cl. CPC A61L 9/20; B60H 3/00; A61L 2209/16; A61L 2209/14; A61L 2209/15; A61L 2209/111; A61L 2209/12

(57) ABSTRACT

A system and method for retrofitting a vehicle with a UV light air sterilization system is installed by forming one or more openings in the ventilation system and sealingly securing UV LED light source units in each of the openings in an air-tight connection so that air does not escape the ventilation system through said openings. The UV lights are connected electrically with the electrical system of the vehicle and that illuminates them at a frequency and intensity of UVC light with germicidal or bactericidal effect on air in their location. A switch or relay switches the electrical current on and supplies electrical current to the UV light sources when manually activated by a user of the vehicle, responsive to electrical power being supplied to the fan device, or responsive to a sensor detecting airflow in the ventilation system.

### RELATED APPLICATIONS

This application claims priority of U.S. provisional application Ser. No. 63/069,642 filed Aug. 24, 2020, which is herein incorporated by reference.

### FIELD OF THE INVENTION

The invention relates to a sterilization of air in vehicles and more particularly to sterilization of air in a vehicle using a UV light source.

### BACKGROUND OF THE INVENTION

Vehicles, and particularly cars, trucks and buses, have enclosed environments that may contain air carrying bacteria or viruses that can endanger the health of persons in the vehicle.

In existing vehicles, the air in these vehicles is normally derived from the outside environment or from recycling air from inside the vehicle, or both combined. The air is then heated or cooled, and supplied through a fan in a fan box in the vehicle.

The air is in some vehicles filtered using a HEPA filter that filters out particulate material that may carry infectious bacteria or viruses. However, the HEPA filter may nonetheless allow passage of some live bacteria or viruses in the air from outside the vehicle. Also, viruses or bacteria may be introduced into the compartment air by infectious passengers in the vehicle, and then that air, when recycled, is distributed by the fan in the fan box, potentially exposing other occupants of the vehicle to the contamination.

### SUMMARY OF INVENTION

It is therefore an object of the invention to provide for sterilization of air in a vehicle compartment. The UV sterilization system sterilizes air being delivered to the passenger compartment of a vehicle, e.g., a car, truck, bus or other vehicle.

It is still further an object of the invention to provide for a method of retro-fitting vehicles with UV-light sterilization of air supplied by their heating or air-conditioning systems.

According to an aspect of the invention, a method of retrofitting a vehicle having a ventilation system and a pre-existing electrical system with a UV light air sterilization system is provided. The method comprises forming an aperture in a portion of the ventilation system of the vehicle communicating with a passage therein through which air passes and is supplied to a passenger compartment o the vehicle, and installing a UV light source in the aperture so as to seal the aperture about it. The UV light source is connected to the electrical system so that the UV light source receives an electrical current from it and is illuminated and generates UV light in an intensity sufficient to provide a germicidal or bactericidal effect. The UV light source is supported in a position and orientation such that UV light generated by the UV light source irradiates air in the passage with at least partial germicidal or bactericidal effect.

UV light is provided by a lamp mounted in the fan box, or just before or after the fan box, of the vehicle or both. Two UV lights may also be placed in, respectively, the intake duct drawing air to the HVAC system of the vehicle and the duct transmitting the heated or cooled air back to the passenger compartment. The intake duct may draw the air from the passenger compartment of the vehicle or from the exterior of the vehicle, or there may be two intake ducts, one drawing air form the interior passenger compartment of the vehicle and the other from the outside. The UV light is of a frequency and power level to sterilize air passing through the fan box or the ducts, preferably after filtration by any HEPA filter or other filtration system.

A kit for retrofit of vehicles comprises a template for placing suitable openings in the fan box, or just before or after the fan box, of a vehicle, as well as mounting structure and sealing structure sealing the UV light in the opening created in the fan box so as to seal the fan box against air leaking out through the added aperture in the fan box wall.

It is also an object of the invention to provide a unit for use in a car passenger compartment that sterilizes the air in the compartment and is powered by the accessory power, e.g., the cigarette lighter power jack or a USB power jack, commonly present in the vehicle.

Other objects and advantages of the invention will be apparent from this specification.

### BRIEF DESCRIPTION OF DRAWINGS

FIG. 1 shows a schematic diagram of a device according to the invention in a vehicle.

FIG. 2 is a detail of the UV light mounted in the wall of the fan box of the vehicle.

FIG. 3 shows the components of a kit supplied for installing a UV-light sterilization system in a vehicle fan box that does not have the capability.

FIG. 4 shows the UV light source component of the kit and the associated template used for installation.

FIG. 5 is a side view detail of part of the HVAC system of a vehicle with the template applied for preparation of the fan box for installation.

FIG. 6 shows a UV light source suitable for use in the invention.

FIG. 7 is a more detailed diagram of the system of FIG. 1.

FIG. 8 is a diagram as in FIG. 7 showing an alternate placement of the UV light source.

FIG. 9 is a diagram as in FIG. 7 showing still another alternate placement of the UV light source.

FIG. 10 is a diagram of the electrical connection of the vehicle power supply to the fan and UV light source in a system of the invention.

FIG. 11 is a diagram of another electrical connection of the vehicle power supply to the fan and UV light source in a system of the invention.

FIG. 12 is a diagram of still another electrical connection of the vehicle power supply to the fan and UV light source in a system of the invention.

FIG. 13 is a diagram of still another electrical connection of the vehicle power supply to the fan and UV light source in a system of the invention.

FIG. 14 is a diagram of a separate UV air sterilization unit for use in a vehicle cabin.

FIG. 15 is a diagram of a retrofit installation of a UV air sterilization system in a vehicle.

### DETAILED DISCLOSURE

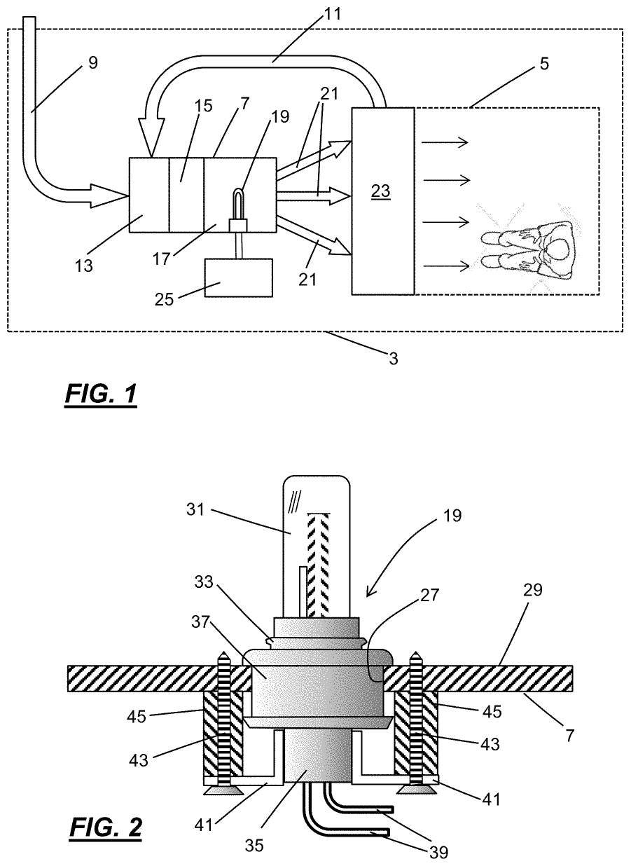

As schematically illustrated in FIG. 1, a vehicle, generally indicated at 3, has a passenger compartment 5 in it in which a driver and passengers are seated or otherwise enclosed. A fan box 7 in the vehicle is connected to a hose or conduit 9 that communicates with the environment and receives air from it. A recycle conduit 11 is connected so as to communicate with the compartment 5 and draw air from it that is supplied to the fan box 7. A control system, commonly known in the art, allows selective control by a user of the flow of air through the conduits 9 and 11. The fan box 7 receives the air from the conduits 9 and/or 11 in portion 13, which contains a fan, not shown, that draws the air in and transmits it through a filter 15 to a chamber 17 in fan box 7.

The chamber 17 is pressurized by a fan (not shown) supported in chamber 17 that draws the air from conduit 9 and/or 11 through the filter, preferably a HEPA filter, and the filtered air is impelled by the fan to flow out of chamber 17 via one our more air supply conduits 21 that deliver the air to the compartment 5 via a vent system generally indicated at 23. The fan box 7 is preferably also equipped with heating or cooling systems to adjust the temperature of the air to a preselected level, and the user can also select which of the conduits 21 should deliver the air to the passenger compartment and where it should flow, as is well known in the art.

Chamber 17 houses a UV light source 19 that generates UV light of a frequency and at an intensity such that the UV light sterilizes the filtered air in the chamber 17 and kills infectious bacteria or viruses in the air. Preferably the intensity of the UV light is sufficient to kill all the bacteria and/or viruses in the air, but even a partial sterilization of the air will reduce the amount of living infectious bacteria or viruses, which is desirable.

The UV light source is connected with the electrical power system of the vehicle, indicated at 25, as will be set out in greater detail below.

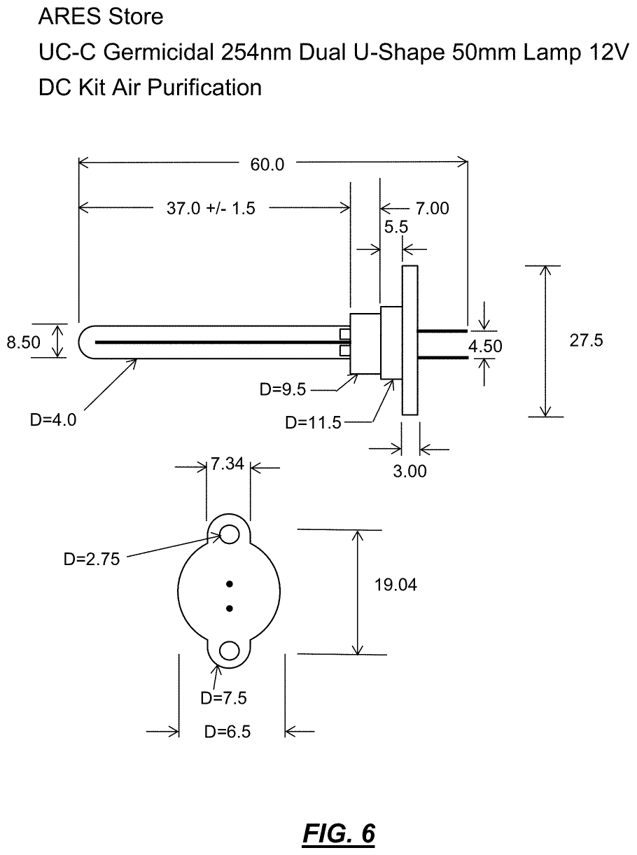

The UV light used for sterilization is in the range of 200 to 300 nm wavelength UV, more preferably 220 to 280 nm, and most ideally around 265 nm wavelength. However, in the preferred embodiment, the UV light source is a commercially available UV-C mercury-vapor bulb well known in the art, and it generates germicidal UV output with a peak intensity at a wavelength of 254 nm (actually 253.7 nm) and drawing 12V DC electrical power. A suitable UV light source of this sort is shown in FIG. 6. The UVC source also does not generate ozone in any significant amounts.

FIG. 2 shows in detail the assembly for installation of the UV light system in a retrofit application where the UV source is installed in a pre-existing fan box in a vehicle.

The UV source 19 is supported in a circular aperture 27 that is formed in installation in the wall 29 of the fan box 7 of the vehicle. The UV source 19 comprises a UV bulb 31 that is screwed into a socket, e.g., E-17 socket in the mounting structure 35 that extends through the wall 29 and through sealing grommet 37. Mounting structure 35 houses wiring 39 that extends through it and connects to the socket 33, supplying electrical power from the vehicle electrical harness.

Mounting structure 35 is fixedly attached to L-shaped brackets 41 affixed thereto that extend outward from it. Screws 43 extend through holes in brackets 41, and through spacer supports 45 to screw into holes in the wall 29, fixedly supporting the apparatus in the wall 29.

The assembly for mounting the UV source in a fan box may vary considerably depending on the clearance available in a given fan box and the angle of placement of the UV bulb relative to the wall of the fan box. The associated mounting structure conforms to the orientation of the UV bulb when installed.

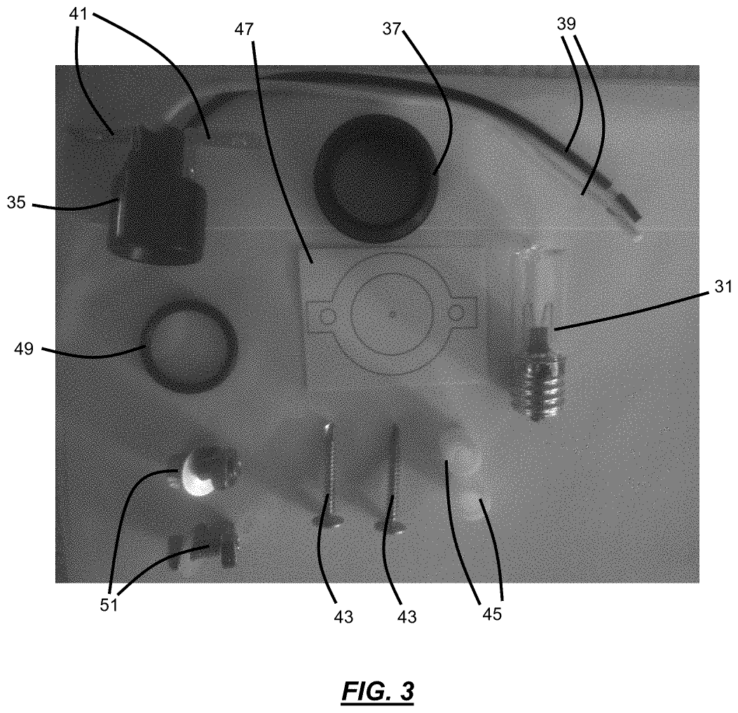

FIG. 3 shows the components of a kit for retrofitting a vehicle with a UV-light sterilization system of the invention. It comprises the bulb 31, the socket/mounting structure 35 with wires 39 and mounting brackets 41, the grommet 37 and screws 43. It further includes a template or stencil 47 that aids in drilling and cutting appropriate openings in the wall of the fan box for installation. It further includes ring 49 and nut, bolt and washer sets 51 that secure and stabilize the E17 socket with the UV-C bulb where it passes through the thin wall of the air filter housing of the fan box. The structure extending through the wall seals off air from escaping from or entering into the fan box through the opening in which the UV light is installed.

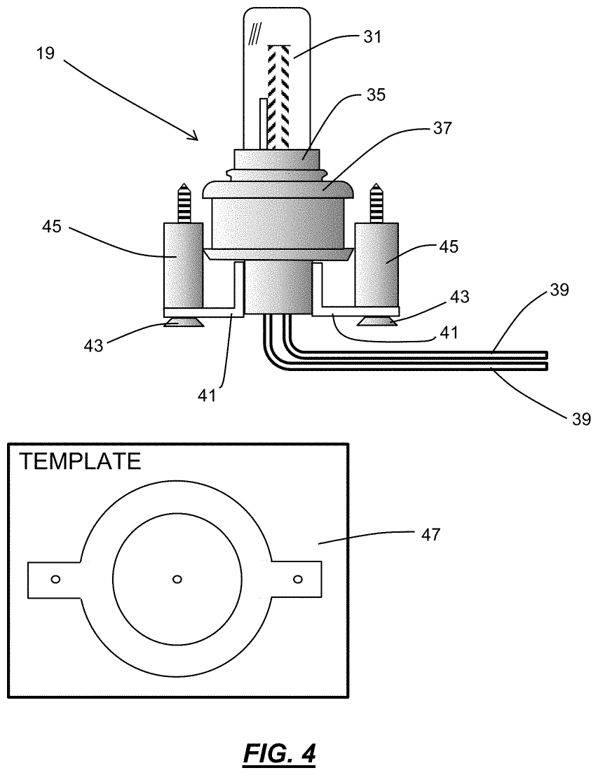

FIG. 4 shows the components of the kit assembled over the template 47.

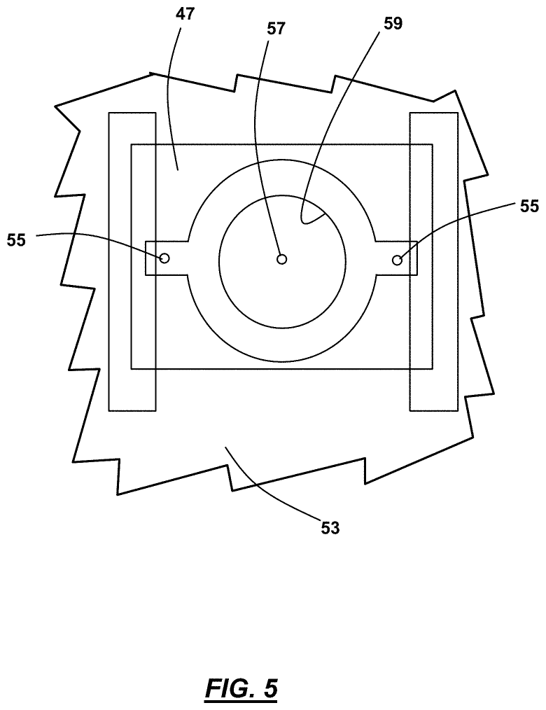

FIG. 5 shows the installation of the UV light using the kit. An exemplary wall portion 53 of the vehicle HVAC system to which the template 47 has been affixed. The wall portion 53 may be in the fan box, or any suitable portion of the vehicle HVAC system or conduits. The template 47 is applied to the flat surface portion 53 by any appropriate method, such as by tape. The wall 53 is part of a housing, and the location of the template is preferably on the discharge side of the filter in the HVAC system. If two UV sources are being installed, the second UV source would employ a second kit the same as the first kit. The location should be selected so that the bulb when mounted in the opening is not obstructed on the inside of the fan box by the filter or any internal structure of the fan box. Also, if there is internal structure of the fan box, the location should not place the UV bulb in a position relative to that interior structure that blocks the radiation of air in the fan box.

When so applied, the template 47 indicates with smaller circles 55 the locations at which the installer drills holes sized to threadingly receive the screws 43.

The template 47 also defines a center point 57 of a larger circle 59 that is dimensioned to match the opening that is cut in the wall to receive the mounting structure 35. Using the center point 57, a drill with a circular cutting tool is used to cut the opening in the wall in the location and with the size of the circle 59.

In the process of installation, first the screw holes and the large circular hole are drilled or otherwise cut in the wall 53. The UV bulb is then screwed into the E-17 socket, and the screws are inserted through the brackets 41 and the support spacers 45, and then screwed into the holes located by circles 55 of the template 47. When so attached, the brackets 41 hold the mounting structure 35 extending through the wall 29 with the bulb 31 inside the housing.

To ensure that the UV light will operate, the wires 39 connected with the E-17 socket are then connected to the vehicle 12 volt DC electrical system. The working of the UV bulb should be confirmed, and then the electrical system may be disconnected to extinguish the bulb during installation.

The fan box usually has two parts of a way of opening the fan box for servicing the filter that it houses. The fan box interior is then opened so as to provide inside access to the UV source 19 and the rubber grommet 37 is fitted over the mounting structure 35 and socket 33 and into the opening 27 in the wall. The grommet 37 sealingly secures the UV source 19 in the wall so that there is no airflow out of the fan box through the opening around the grommet 37 or the mounting structure 35, maintaining the pressure in the fan box as though there were no UV source.

Once so installed, the electrical system is re-connected to the UV source wires 39, illuminating the UV bulb 31. The interior of the fan box is then continuously irradiated so as to sterilize air flowing through it into the compartment 5.

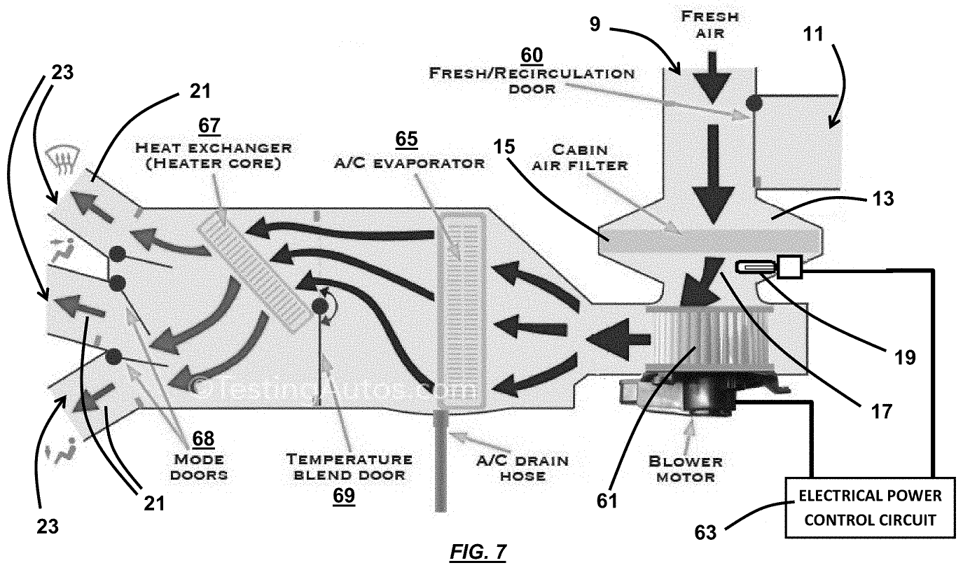

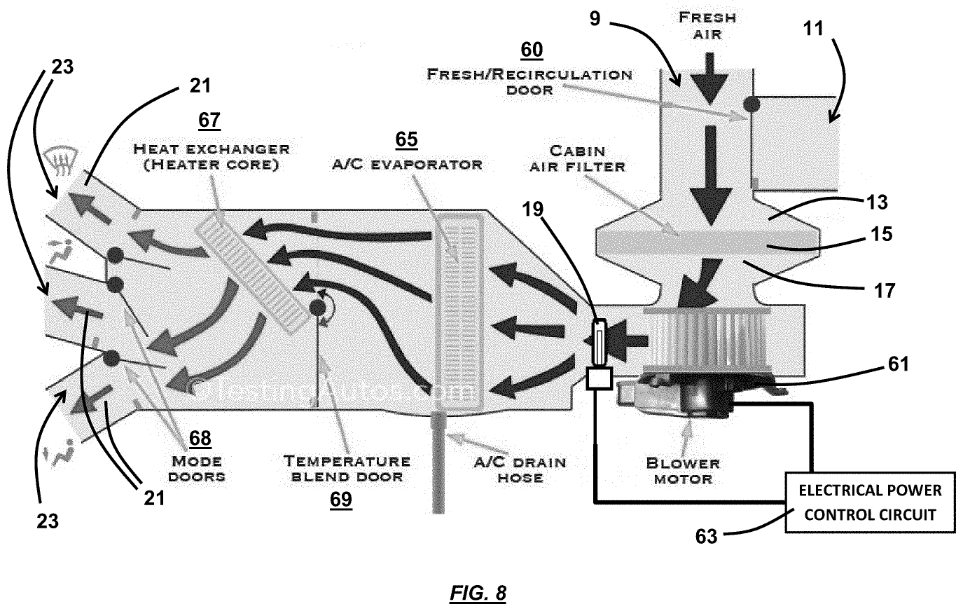

FIG. 7 shows details of the structural integration of the UV light source into the HVAC of a vehicle, especially an automobile. The same reference numbers are used for the same parts as schematically illustrated in FIG. 1.

Referring to FIG. 7, an electric blower motor 61 is powered by 12 volt DC current from the automobile electrical harness power circuitry 63. When powered and operating, the blower motor 61 draws air in from conduits 9 and/or 11 so that it passes through HEPA filter 15, which filters out small particulate contaminants in the air passing through it. The source of the air is selected by door 60, which selectively covers either conduit 9 or 11, or neither, and controls whether outside air, recirculated air or both are supplied to the HVAC system.

The motor 61 operates at varying speeds controlled by the user of the vehicle, usually by a user-controlled potentiometer in circuitry 63 that sets the voltage of current supplied to the motor 61, resulting in different user-selected speeds of the motor and the force and speed of air flow from the HVAC system. The air from the blower fan 61 flows through an air conditioner evaporator coil 65 and through a heater core 67, which together are controlled by the user by mechanisms including temperature blend door 69 that mixes the cooled and heated air in user selected amounts ranging from 100% cooled air to 100% heated air, as is well-known in the art. The air having the user selected temperature is then blown through mode doors 68 that are open or closed to select where the air is supplied in the compartment of the vehicle through conduits 21 and the relevant selected vents 23.

UV light filter 19 in the preferred embodiment is placed in the fan box in a location after the HEPA filter 15 and before the fan 61. The irradiated air is there generally free of particulate material that could provide a place for microorganisms to hide and conceal themselves from the germicidal UV light. The UV light is electrically connected with the electrical power control circuitry 63 and is powered by it to illuminate the UV lamp to germicidal levels of UV output. The connection is such that the UV light 19 is illuminated by transmitting a 12 volt DC current form the car electrical harness when the motor 61 is turned on.

FIG. 8 shows an alternate embodiment of placement of the UV light source 19 in the vehicle. If clearance permits it, the UV lamp may alternatively be placed in the fan box wall to sterilize he air flow after it passes through the fan 61.

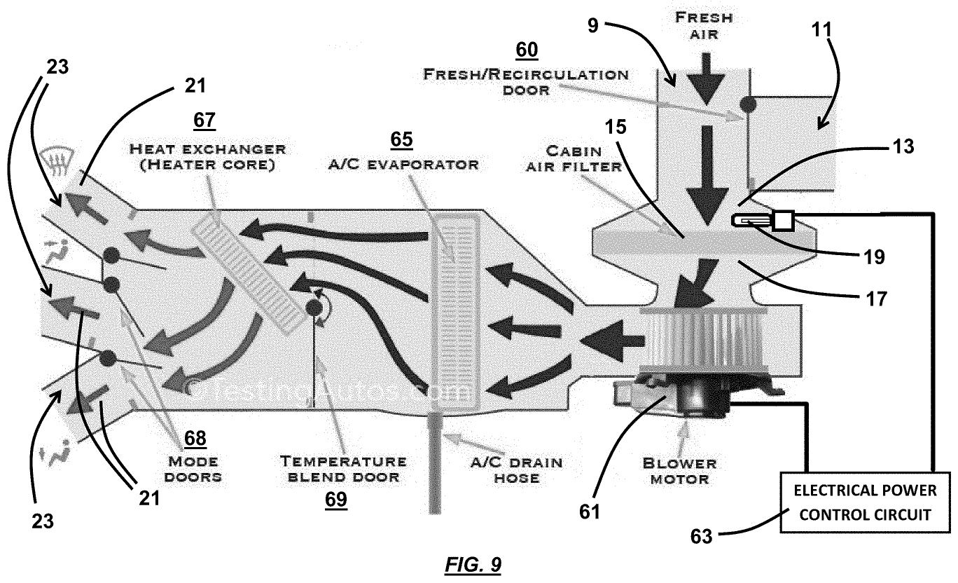

FIG. 9 shows still another alternate embodiment of placement of the UV light source 19 in the vehicle. If clearance permits it, the UV lamp may be placed in the fan box wall in the portion 13 of the chamber before the air flow passes through the filter 15. In this position, the UV light 19 still sterilizes the air flow to some degree, including the sterilization at least to a degree of the particulate matter that will be removed by filter 15.

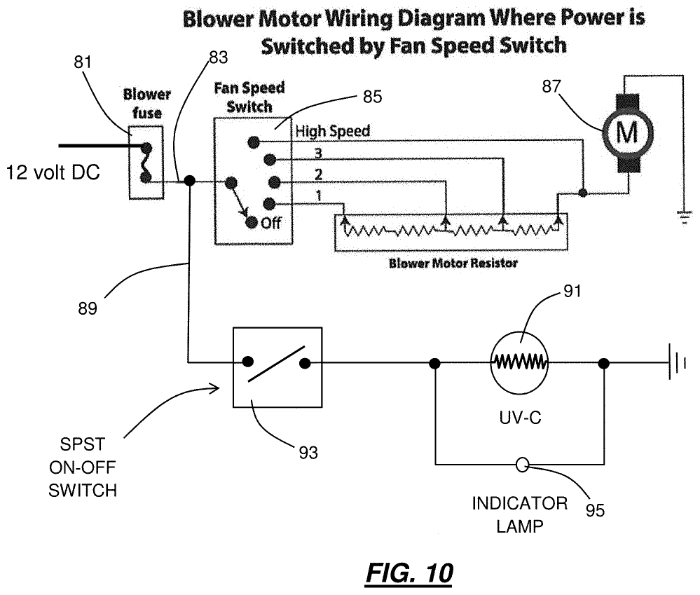

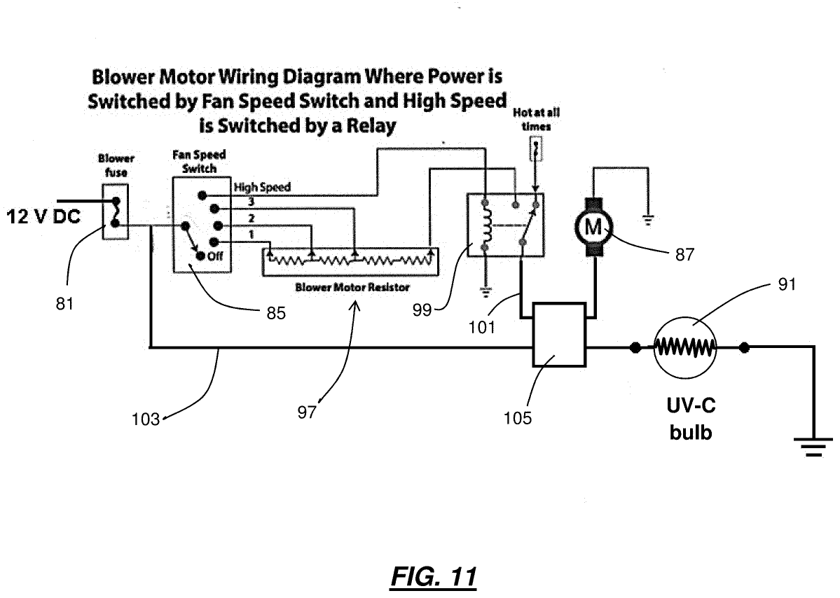

In the preferred embodiment, the UV light 19 is powered and illuminated only when the fan motor 61 is powered to create air flow, and it is left off when the fan motor 61 is not powered and is not operating. This is illustrated in the diagram of the connected circuitry shown in FIG. 10.

An automobile commonly has an electrical system with a battery and an alternator that supplies DC current for various functions of the vehicle and its accessories. Generally, the DC current is supplied by the electrical system, indicated at 71, as 12 volt DC. That current is supplied in the vehicle to the HVAC system, which has a switch 73 that turns on the fan 61 when a user of the vehicle turns on the HVAC.

The HVAC system usually also includes a speed control 75 for the fan that allows the user to set the force of the air flow, e.g., to low, medium and high speeds. The control usually is a potentiometer that adjusts the voltage of DC current sent to the fan motor 61, responsive to which the fan runs faster or slower, as selected. Notwithstanding the variations in power supplied to the fan motor, the full 12 volts of DV power is supplied to the UV lamp, causing it to be illuminated at full germicidal intensity when the fan is on, and turning it off when the fan is off.

If the sole on/off control for the HVAC motor is the potentiometer, then special circuitry is provided that is configured to transmit the 12 volt DC current to the UV light irrespective of the voltage supplied to the fan motor.

FIG. 10 shows a diagram of a circuit that can be used to retrofit a vehicle with the UV light sterilization system as described above. The 12 volt DC current power from the automobile or vehicle electrical harness is supplied to a blower fuse 81 from which wiring 83 connects to a fan speed switch that allows a user to adjust the speed of the fan blower motor 87. The fan speed switch is a potentiometer that applies selective resistance to control the motor speed, with full 12 volt voltage current being applied at high speed selected, and slower speeds with reduced voltages, as is well known in the art.

In the retrofit installation of the UV light, the light source, the UV light source 91 is installed in the fan box or other appropriate location of the HVAC system. A wire 89 is connected with the wiring 83 so as to draw the 12 volt current. The wire 89 leads to a manual switch 93 that is positioned in the vehicle, such as on or adjacent the car dashboard, so that it can be accessed and operated by a user to turn on the UV sterilization light 91. When switched on, the 12 volt current flows to the UV light source 91 which is connected at its other side to vehicle ground, causing it to illuminate and sterilize the air in the HVAC system.

An indicator lamp LED 95 is wired in parallel with the UV lamp 91, and generates visible light when the UV light source 91 is illuminated.

FIG. 11 shows an alternate circuit for retrofit, with similar parts indicated with the same reference numbers. The 12 volt DC current from the blower fuse 81 flows to the operator speed control 85, which has a circuitry 97 that supplies power at variable voltages, up to a peak high-speed voltage applied by a solenoid relay 99. The varied voltage power DC current passes through wire 101 and flows to motor 87, which is connected at its other side to vehicle ground, causing the motor to turn at a speed corresponding to the selected voltage.

During retrofit installation, a wire 103 is connected to receive the 12 volt DC current and carry it to a switch 105. Switch 105 is a relay that is activated and closed whenever there is current of any voltage passing through conductor 101, allowing 12 volt current to flow to UV source 91 to vehicle ground. As a result, when the motor 87 is operating at any speed, the UV light source 91 is illuminated at full operating intensity, and when it is off, the UV light 91 is also off.

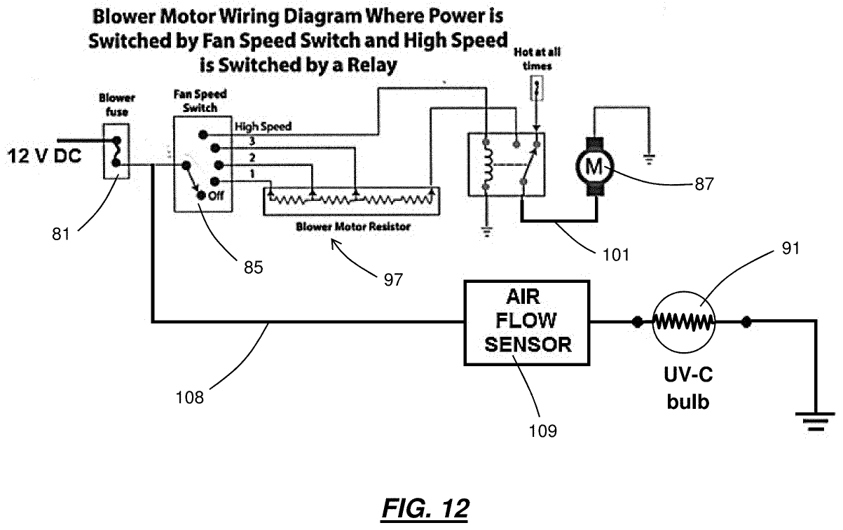

FIG. 12 shows another embodiment of retrofit circuit, similar to the circuit of FIG. 11. Wire 108 receives the DC current and takes it to an airflow sensor switch 109. Air flow sensor 109 closes the circuit responsive to sensing flow of air in the HVAC system resulting from operation of the fan motor. When the circuit is closed, the 12 volt current flows through UV bulb 91 and to vehicle ground, causing it to illuminate and sterilize the air in the HVAC system.

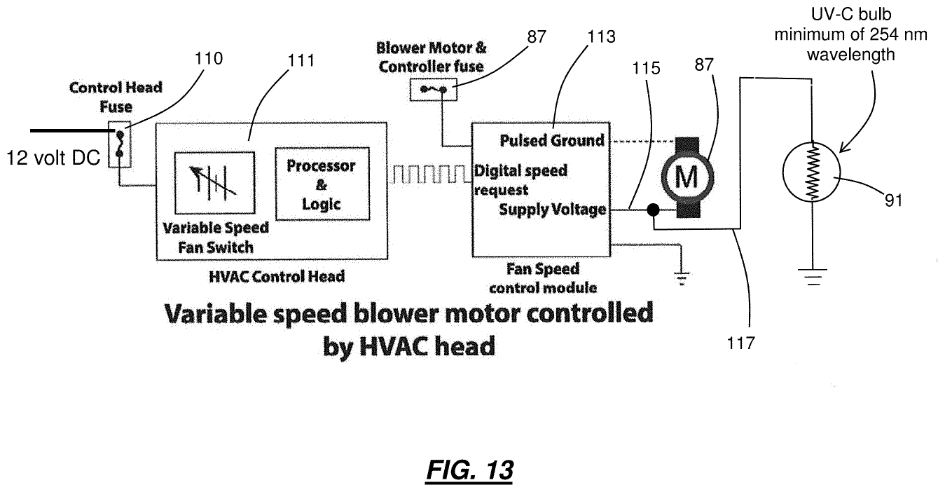

FIG. 13 shows still another circuit that may be used in retrofit where the vehicle relies on a pulsed connection to adjust the speed of the fan motor. The 12 volt DC current is provided through a fuse 110 to a logic circuit component 111 that has an operator adjustable fan speed control. The component outputs a pulsed wave that indicates a desired motor speed. Motor control 113 receives the 12 volt current from fuse 18 and supplies it to the fan motor 87 via wire 115. The motor control 113 also cyclically connects the opposite side of the motor 87 to ground, which reduces net voltage over time to the motor 87 and sets its speed. A wire 117 is connected to the full 12 volt current line 115, and supplies power to the UVC bulb 91, the other side of which is connected without pulsing to vehicle ground, so that the UV light is illuminated when the motor 87 is being powered to run at any speed.

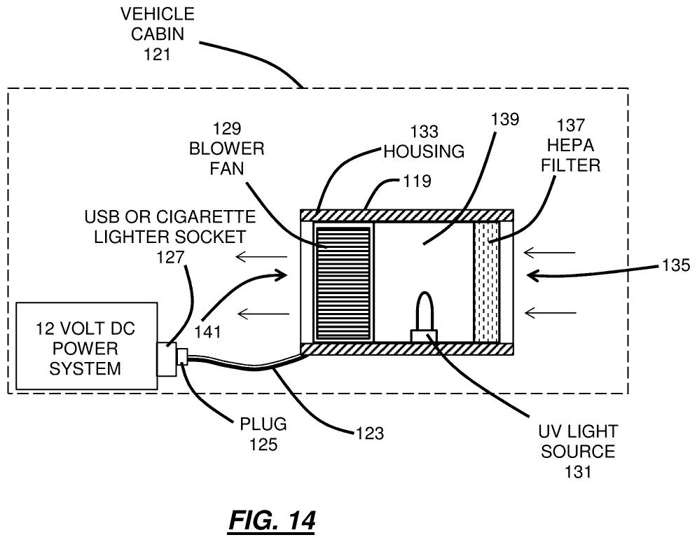

FIG. 14 shows another embodiment of UVC air sterilizer for a vehicle. A modular unit 119 is placed in the passenger compartment or cabin 121 of the vehicle. It is powered by connection of cable, which has a plug 127 configured to be operatively received in the USB or cigarette lighter socket 127 of the automobile and to receive 12 volt dc current from it. A manually operated switch may be included in the cable 123.

The cable extends to the modular unit 119 and is connected to power a blower fan 129 and a UVC light source 131, which is similar to the one described in previous embodiments. When powered, the blower fan 129 draws air through the housing 133 of unit 119 from an inlet side indicated at 135 and through HEPA filter 137 into sterilization chamber 139. In the chamber 139, UVC bulb 131 is continuously illuminated while the blower is operative, sterilizing the air as it passes through to blower fan 129. The UV light source is surrounded by opaque shield structure such that UV light cannot escape the chamber 139. Blower fan 129 then expels the sterilized air through the unit outlet side 141.

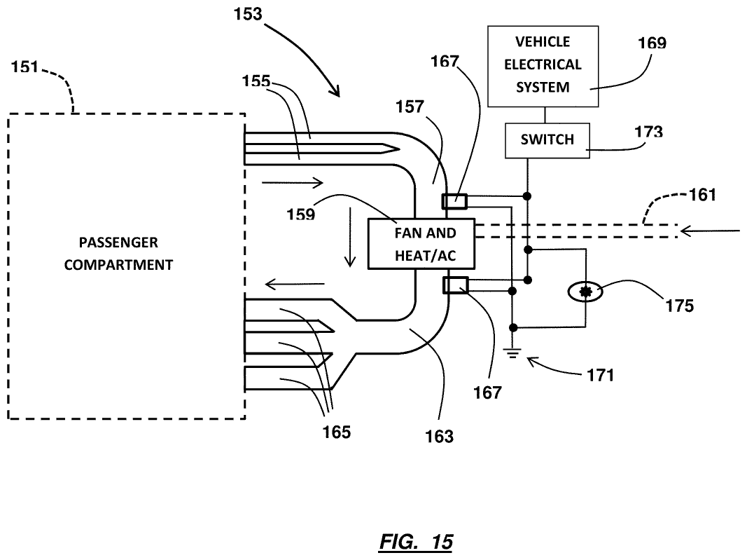

FIG. 15 is a diagram showing a retrofit installation of UV light air sterilization in a vehicle as well known in the art, having a passenger compartment 151 connected with a ventilation system generally indicated at 153, which has one or more intakes 155 leading to a combined intake conduit 157 that carries the air drawn in through the intakes 155 to a fan box or fan and heating and/or sir conditioning system 159 that has an electrically powered fan device, as has been described above, as well as heating or cooling devices that heat or cool air supplied by the fan box 159. The fan system 159 is connected with an inlet 161 that selectively admits air from the external environment and the fan system 159 has structure well known in the art and as described above that provides for selection of whether the air heated or cooled and output by the fan system 159 is recycled air from the passenger compartment, air from outside the vehicle, or a mixture of those two. The pre-existing vehicle ventilation system has an output conduit 163 receiving air from the fan system 159 and transmitting it through vents 165 to various portions of the passenger compartment 151, as is also well known in the art.

The retrofit of the vehicle to provide it with UV light sterilization involves installation of preferably two or more UV light sources in the ventilation system allowing for repeated UV light exposure of air passing through the system so as to provide a maximal sterilizing effect on the air supplied to the passenger compartment 151. The locations of installation may vary, provided that the installed UV light sources provide enough radiation to the air passing through the ventilation system in the location to provide at least some germicidal or bactericidal effect from the UV, and provided that the UV light source has adequate clearance inside the location in the ventilation system 153 such that it does not materially interfere with airflow in that location.

The location or locations may be any of the locations of installation shown in FIG. 7, 8 or 9, or may be in the intake or outlet conduits that connect the passenger compartment to the fan system 157. There may be issues with internal space for the UV light sources.

This is partially resolved by providing UV light sources that require less space and are desirable for energy considerations as well. The UV light source may be a germicidal UV sanitizer light bulb requiring 25 W current and generating UV light with a frequency of 254 nm. More preferred, however, is a UV light source with an LED generating UV light, such as the LED present in the UV antiviral light bolt flush mount running on 12V DC current and incorporating an ultraviolet UVC germicidal LED that is used to sanitize or disinfect the interior, footwell, or trunk of cars, sold by Oznium.com, 135 Country Center Dr., Ste. F #242, Pagosa Springs, Colo. 81147. Other LEDs that may be employed are those found in higher energy consumption UV light sources or UV light sanitizer lamps that contain a substantial number of LEDs that generate UVC light with a wavelength of 260-280 nm.

As best seen in FIG. 15, especially where a low profile LED UV light source is used, UV light sources 167 may be installed in the inlet conduit 157 and the output conduit 163. This is accomplished by forming an opening or aperture in the relevant conduit that accommodates the UV light source unit 167 and receives it sealingly therein, with an airtight seal such as the seal described previously in, e.g., FIG. 2. In these locations, the UV light sources 167 each can irradiate air passing through the associated conduit, which cumulatively substantially sterilizes the air provided to the passenger compartment.

After installation of the sources 167 in the conduits or fan box, each of the UV light sources 167 is electrically connected with the vehicle electrical system 169, which supplies electrical current sufficient to cause the UV lights 167 to illuminate and generate UV light at a frequency and an intensity that has germicidal or bactericidal effect on air passing through the associated conduit, generally being DC current at 12V or 24V. The UV light sources 167 are also connected at another side to vehicle ground 171 to complete the circuit.

Switch 173 is provided in the connection between the vehicle electrical system 169 and the UV lights 167. Switch 173 selectively supplies the requisite electrical current from the electrical system 173 to the UV lights, and may be a manual switch as shown in FIG. 10, a relay that switches on the electrical current when power is supplied to the fan motor in the fan system 159 as shown in FIGS. 11 and 13, or when an airflow sensor in the associated conduit indicates that air is flowing through it (see FIG. 12).

The switch 173 may supply current to the UV light sources 167 in parallel so that they are switched on and off together. Alternatively, the switch 173 may comprise a plurality of switches, each supplying power to a respective UV light source 167 so that it may be illuminated independently when switched on manually by a user or automatically responsive to activation of the relay or airflow sensor.

Preferably an indicator light 175 is present that illuminates with visible light when electrical current is flowing to the UV lights so that a user can determine if the UV sterilization system is turned on or not.

This retrofit of the vehicle is accomplished without substantially impacting normal operation of the vehicle ventilation system. As a result of the installation, when the UV light sources are turned on, air recirculated from the passenger compartment is subjected to at least two exposures to germicidal or bactericidal UV light, producing a healthier environment in the vehicle.

The terms herein should be read as terms of description rather than limitation, as those of skill in the art with this disclosure before them will be able to make modifications therein without departing from the spirit of the invention.

What is claimed is:

1. A method of retrofitting a vehicle having a ventilation system and a pre-existing electrical system with a UV light air sterilization system, said method comprising:
forming an aperture in a portion of the ventilation system of the vehicle communicating with a passage therein through which air passes and is supplied to a passenger compartment o the vehicle;
installing a UV light source in the aperture so as to seal the aperture thereabout;
connecting the UV light source to the electrical system so that the UV light source receives an electrical current therefrom and is illuminated and generates UV light in an intensity sufficient to provide a germicidal or bactericidal effect;
the UV light source being supported in a position and orientation such that UV light generated by said UV light source irradiates air in the passage with at least partial germicidal or bactericidal effect.

2. The method of claim 1, and further comprising
forming a second aperture in a portion of the ventilation system displaced from the first portion;
installing a second UV light source in the aperture so as to seal the second aperture thereabout;
connecting the second UV light source to the electrical system so that the UV light source receives an electrical current therefrom and is illuminated and generates UV light in an intensity sufficient to provide a germicidal or bactericidal effect;
wherein the ventilation system has a fan device drawing air from the passenger compartment thorough an intake conduit and delivering air from the fan device via an output conduit to the passenger compartment, the first and second UV light sources being supported in the intake conduit and the output conduit, respectively, such that air drawn from and returned to the passenger compartment is subjected to germicidal or bactericidal radiation form both of the UV light sources.

3. The method of claim 2, wherein the UV light sources each comprises an LED generating UV light.

4. The method of claim 1, wherein the ventilation system has a fan device drawing air therethrough, said fan device being powered by electrical current received from the electrical system of the vehicle; and
said UV light source being connected to the electrical system by an electrical connection in parallel with the fan device.

5. The method of claim 4, wherein the electrical connection includes a switch that selectively causes the electrical current to flow to the UV light source and illuminate the UV light source so as to sterilize air in the ventilation system.

6. The method of claim 5, wherein the switch is a manual switch configured to be operated selectively by a human user.

7. The method of claim 5, wherein the switch is connected with an air flow detector that detects flow of air in the ventilation system, and the switch causes supply of the electrical current to the UV light source responsive to the air flow detector detecting flow of air in the ventilation system, and switches off the flow of electrical current to the UV light source when the air flow detector does not detect flow of air in the ventilation system.

8. The method of claim 5, wherein the switch is a relay that supplies the electrical current to the UV light source at a predetermined constant operating voltage responsive to electrical current being supplied to the fan device at any voltage level, and switches off the electrical current to the UV light source when no electrical power is being supplied to the fan device.

9. The method of claim 5, wherein an indicator light is connected with the UV light source, said indicator light generating visible light so as to visible to a user when the UV light source when the UV light source is generating UV light.

10. The method of claim 1, wherein the ventilation system has a filter filtering air passing therethrough, and the UV light source is placed in the ventilation system so as to irradiate air after it passes through the filter.

11. A method of retrofitting a vehicle with a UV light air sterilization system, wherein the vehicle has a ventilation system with an intake passage and an outlet passage communicating with an interior passenger compartment of the vehicle, and a fan device powered by an electrical system of the vehicle and that draws air through the intake passage and directs the air through the outlet passage to the interior passenger compartment after heating or cooling thereof, said method comprising:
forming a plurality of openings in the ventilation system;
sealingly securing UV light source units each in a respective one of the openings in an airtight connection so that air does not escape the ventilation system through said openings; and
connecting each of the UV light source units electrically with the electrical system so that electrical current flows thereto and causes illumination thereof at a frequency of UV light and at an intensity such as to produce germicidal or bactericidal effect on air in a location around the UV light;
wherein a switch is provided between the UV lights and the electrical system that selectively switches the electrical current on at a power level sufficient to produce a sterilizing effect from the UV light sources, said switch comprising a switch element that supplies electrical current to the UV light sources responsive to manual activation by a user of the vehicle, to electrical power being supplied to the fan device, or to a sensor detecting airflow in the ventilation system.

12. The method according to claim 11, wherein the UV light sources are each installed in a respective location, one of said locations being in the intake conduit and the other of the locations being in the outlet conduit such that air from the passenger compartment passing through the ventilation system is irradiated twice when the UV lights sources are illuminated.

13. The method of claim 11, wherein a visible light indicator lamp is connected with the UV lights and the indicator light generates visible light when the UV light sources are powered and generating UV light.

14. The method of claim 11, wherein each of the UV light sources is an LED that generates UVC light at germicidal intensity with a wavelength in the range of 260 to 280 nm.
